(19) United States
(12) Patent Application Publication
(10) Pub. No.: US 2019/0056707 A1
(43) Pub. Date: Feb. 21, 2019

(54) Methods and Systems for Controlling Operation of Equipment Based on Biometric Data

(71) Applicant: The Boeing Company, Chicago, IL (US)

(72) Inventors: Gerald Pollard, Chicago, IL (US); James Lynn, Chicago, IL (US)

(21) Appl. No.: 15/680,519

(22) Filed: Aug. 18, 2017

(51) Int. Cl. G05B 19/048; G06F 3/01; G05B 19/46; A61B 5/0205; B64C 25/08; B64F 5/60

(52) U.S. Cl. CPC G05B 19/048; G06F 3/015; G05B 19/46; G06F 3/147; B64C 25/08; B64F 5/60; A61B 5/02055

(57) ABSTRACT

Example methods and systems for controlling operation of equipment are described. An example method for pausing operation of one or more of electrical and hydraulic systems of an equipment includes receiving, at a computing device having one or more processors, biometric data from a wearable biometric interface of a wearer, and based on the biometric data being outside of a range of baseline data, the computing device controlling operation of a power unit coupled to the equipment to pause movement of one or more components of the one or more of electrical and hydraulic systems of the equipment.

### FIELD

The present disclosure generally relates to methods and systems for controlling operation of equipment based on biometric data received from a wearable biometric interface of a wearer, and more particularly to pausing operation of electrical and/or hydraulic systems of an equipment due to received biometric data being outside of a range of baseline data.

### BACKGROUND

In many industrial sites, manufacturing facilities, or other areas involving moving equipment, mechanics and other personnel can be exposed to risk by working near the moving or energized equipment or systems. There are typically two primary methods used today for safeguarding personnel. A first method includes use of cameras to provide real time images of areas where operators are working, and a second method includes use of observers to watch the areas to ensure safety procedures are being followed.

However, equipment and/or other personnel can obscure a view from the camera or observer. This can lead to a mechanic entering a hazardous area to perform a test or inspection without notice to the observer. In other instances, when two different groups of people are working on a same part of equipment and have control of moving components, sometimes problems can arise when communications between the two groups fail leading to possible dangerous conditions being created.

Thus, improvements are desired to further ensure safe working conditions in these areas.

### SUMMARY

In an example, a method for pausing operation of one or more of electrical and hydraulic systems of an equipment is described. A power unit is coupled to the equipment for enabling movement of one or more components of the one or more of electrical and hydraulic systems of the equipment. The method comprises receiving, at a computing device having one or more processors, biometric data from a wearable biometric interface of a wearer. The method also comprises based on the biometric data being outside of a range of baseline data, the computing device controlling operation of the power unit to pause movement of the one or more components of the one or more of electrical and hydraulic systems of the equipment.

In another example, a system is described comprising a wearable biometric interface for sensing biometric data of a wearer, a power unit coupled to equipment for enabling movement of one or more components of the equipment, and a computing device having one or more processors. The computing device is in communication with the power unit, and the computing device receives the biometric data from the wearable biometric interface and controls operation of the power unit, based on the biometric data, for operating one or more of electrical and hydraulic systems of the equipment. The computing device controls operation of the power unit to pause movement of the one or more components of the equipment based on the biometric data being outside of a range of baseline data.

In another example, another system is described comprising a wearable biometric interface having a plurality of biometric sensors for sensing different biometric data of a wearer and one or more environmental sensors for sensing one or more characteristics of an environment of the wearable biometric interface. The system also comprises a computing device, having one or more processors, being in communication with a power unit that is coupled to an equipment for enabling movement of one or more components of the equipment. The computing device receives the biometric data and the one or more characteristics of the environment from the wearable biometric interface and controls operation of the power unit to pause movement of the one or more components of the equipment based on a combination of outputs of (i) the plurality of biometric sensors being outside of a range of baseline data and (ii) the one or more environmental sensors being outside of a range of normal operating conditions.

The features, functions, and advantages that have been discussed can be achieved independently in various examples or may be combined in yet other examples further details of which can be seen with reference to the following description and drawings.

### BRIEF DESCRIPTION OF THE FIGURES

The novel features believed characteristic of the illustrative examples are set forth in the appended claims. The illustrative examples, however, as well as a preferred mode of use, further objectives and descriptions thereof, will best be understood by reference to the following detailed description of an illustrative example of the present disclosure when read in conjunction with the accompanying drawings, wherein:

FIG. 1 is a block diagram of a system, according to an example implementation.

FIG. 2 is a block diagram of an example of the biometric interface, according to an example implementation.

FIG. 3 is a block diagram of an example of the equipment interface, according to an example implementation.

FIG. 4 is a block diagram of an example of the central monitoring station, according to an example implementation.

FIG. 5 is a block diagram of an example of the computing device, according to an example implementation.

FIG. 6 is a block diagram illustrating an example operation and equipment interface to computing device connection, according to an example implementation.

FIG. 7 is a truth table illustrating an example of the computing device processing the data for controlling operation of the equipment, according to an example implementation.

FIG. 8 is a block diagram illustrating an example work zone environment, according to an example implementation.

FIG. 9 shows a flowchart of an example method for pausing operation of one or more of the electrical system and the hydraulic system of the equipment, according to an example implementation.

FIG. 10 shows a flowchart of an example method for use with the method shown in FIG. 9, according to an example implementation.

FIG. 11 shows a flowchart of an example method for use with the method shown in FIG. 9, according to an example implementation.

### DETAILED DESCRIPTION

Disclosed examples will now be described more fully hereinafter with reference to the accompanying drawings, in which some, but not all of the disclosed examples are shown. Indeed, several different examples may be described and should not be construed as limited to the examples set forth herein. Rather, these examples are described so that this disclosure will be thorough and complete and will fully convey the scope of the disclosure to those skilled in the art.

Within examples described herein, methods and systems for pausing operation of one or more of electrical and hydraulic systems of an equipment. A power unit is coupled to the equipment for enabling movement of one or more components of the one or more of electrical and hydraulic systems of the equipment, and a computing device receives biometric data from a wearable biometric interface of a wearer, and based on the biometric data being outside of a range of baseline data, the computing device controls operation of the power unit to pause movement of the one or more components of the one or more of electrical and hydraulic systems of the equipment. The computing device monitors key biometric parameters of the wearer (e.g., heart rate, blood pressure, galvanic skin resistance, skin temperature, etc.) to determine when a wearer (e.g., mechanic) is likely experiencing sudden and profound stress and immediately takes action to eliminate the risk by pausing movement of the components. In further examples, position sensors provide real-time location of the mechanic relative to danger zones and trigger area alarms and safeguarding actions whenever a mechanic enters a dangerous area.

Bluetooth transceivers in the area using state of the art encryption technology can be programmed to continuously transmit safety status information to system interface controllers and area safety zone monitors. Intelligent application software allows users to quickly and easily customize zone maps, interface parameters, start/stop actions, and other parametric data. Optional data recorders worn by mechanics can provide data logging of real-time status and events and can store up to six months data, for example.

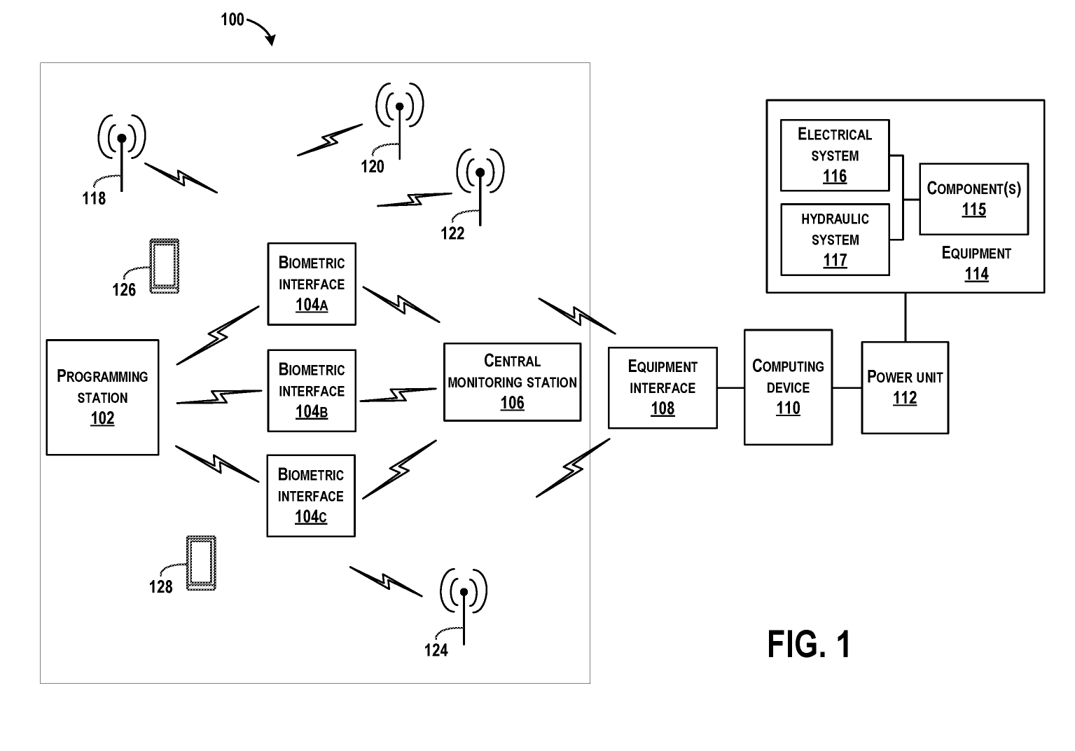

Referring now to the figures, FIG. 1 is a block diagram of a system 100, according to an example implementation. The system 100 includes a programming station 102, biometric interfaces 104a-c, a central monitoring station 106, an equipment interface 108, a computing device 110, a power unit 112, and equipment 114 having components 115. The computing device 110 receives information from components of the system 100, and controls operation of the equipment 114 based on the information received.

The programming station 102 is in communication with the biometric interfaces 104a-c, such as through wireless communication, and the programming station 102 is used to define parameters of the system 100 and to program the biometric interfaces 104a-c before they are distributed to personnel.

The biometric interfaces 104a-c may take the form of a wearable biometric interface that has a plurality of biometric sensors for sensing different biometric data of a wearer, and can also include one or more environmental sensors for sensing one or more characteristics of an environment of the wearable biometric interface.

The central monitoring station 106 is also in communication with the biometric interfaces 104a-c, such as through wireless communication, and the central monitoring station 106 receives data from the biometric interfaces 104a-c, and can display and activate alarms in the system 100 based on the received data. The central monitoring station 106 also can display x, y, z, coordinates of the biometric interfaces 104a-c, adjustments for biometric data thresholds, and voice monitoring and control as well of the biometric interfaces 104a-c.

The equipment interface 108 is also in communication with the biometric interfaces 104a-c, such as through wireless communication, and the equipment interface 108 receives interlock, stop/start status, and other data from the biometric interfaces 104a-c. The equipment interface 108 then provides an interface between the biometric interface 108 and the existing equipment controllers, such as the computing device 110, for example, based on the received data.

The computing device 110 has one or more processors and receives the biometric data output from the wearable biometric interfaces 104a-c, for example via the equipment interface 108, and controls operation of the power unit 112 based on the biometric data. Thus, the computing device 110 is in communication with the power unit 112 for operating one or more of an electrical system 116 and/or a hydraulic system 117 of the equipment 114. Within examples, as described below, the computing device 110 includes data storage storing instructions executable by one or more processors for controlling operation of the power unit 112 for operating of one or more of the electrical system 116 and the hydraulic system 117 of the equipment 114 based on the biometric data. For instance, the computing device 110 controls operation of the power unit 112 to pause movement of the one or more components 115 of the equipment 114 based on the biometric data being outside of a range of baseline data. The computing device 110 also controls operation of the power unit 112 to enable movement of the components 115 of the equipment 114, such as for example, based on outputs of any of the biometric interfaces 104a-c. Further, operation can be controlled based on a combination of outputs received from one biometric interface including a combination of outputs of (i) biometric sensors being outside of a range of baseline data and (ii) environmental sensors being outside of a range of normal operating conditions.

Although FIG. 1 shows the equipment interface 108 and the computing device 110 as separate components, the equipment interface 108 and the computing device 110 can be included within one computing device in the system 100, such as within a test cart, for example.

The power unit 112 is coupled to the equipment 114 for enabling movement of the components 115 of the equipment 114. Within examples described herein, the equipment 114 includes an airplane, and the components 115 include a surface of the airplane and landing gear of the airplane that are under test for operation. However, the system 100 is adaptable to any technology that involves energized or moving components. This includes the airline, automotive, shipping, entertainment, and agriculture industries. Thus, the equipment 114, more generally, can include any machinery (e.g., robot work cells, or other manufacturing processes), and the components 115 of the equipment 114 can include any moving part of the equipment 114.

The system 100 is also shown to include position transceivers 118, 120, 122, and 124 throughout an area that are in wireless communication with the biometric interfaces 104a-c. The position transceivers 118, 120, 122, and 124 provide an ability to determine a position of the biometric interfaces 104a-c when global positioning sensors (GPS) are not available for the biometric interfaces 104a-c, for example. The position transceivers 118, 120, 122, and 124 also may determine positions or locations of other devices engaged in wireless communications in the area, such as device 126 or device 128, so as to locate potential personnel that may be in the area, and operations of the system 100 can be paused or disabled when such unidentified personnel have entered the area, for example.

In an example operation of the system 100, each mechanic wears one of the biometric interfaces 104a-c. The biometric interfaces 104a-c perform biometric sensing, position, interlock status, and stop/start status locally (within each biometric interface 104a-c) with results wirelessly transmitted to the equipment interface 108 and the central monitoring station 106. The biometric interfaces 104a-c thus connect biometric data of the mechanic to the computing device 110 that controls operation of movement of the components 115 of the equipment 114. The computing device 110 will control movement of equipment surfaces, raise and lower gear, and test functions as programmed during testing procedures of the equipment 114. Hydraulic power for the equipment 114 is produced by the hydraulic system 117 and maintains pressure on the equipment 114 while functional tests are being conducted. In any instance in which the computing device 110 receives biometric data that is outside of baseline ranges expected to be seen or experienced by the mechanics, the computing device 110 will cause operation of the equipment 114 to pause as a safeguard for the mechanics that are in areas assisting with the testing of the equipment 114. Alternatively, in instances in which the computing device 110 receives biometric data that is within baseline ranges expected to be seen or experienced by the mechanics, the computing device 110 will control operation of the power unit 112 to enable movement of the components 115 of the equipment 114.

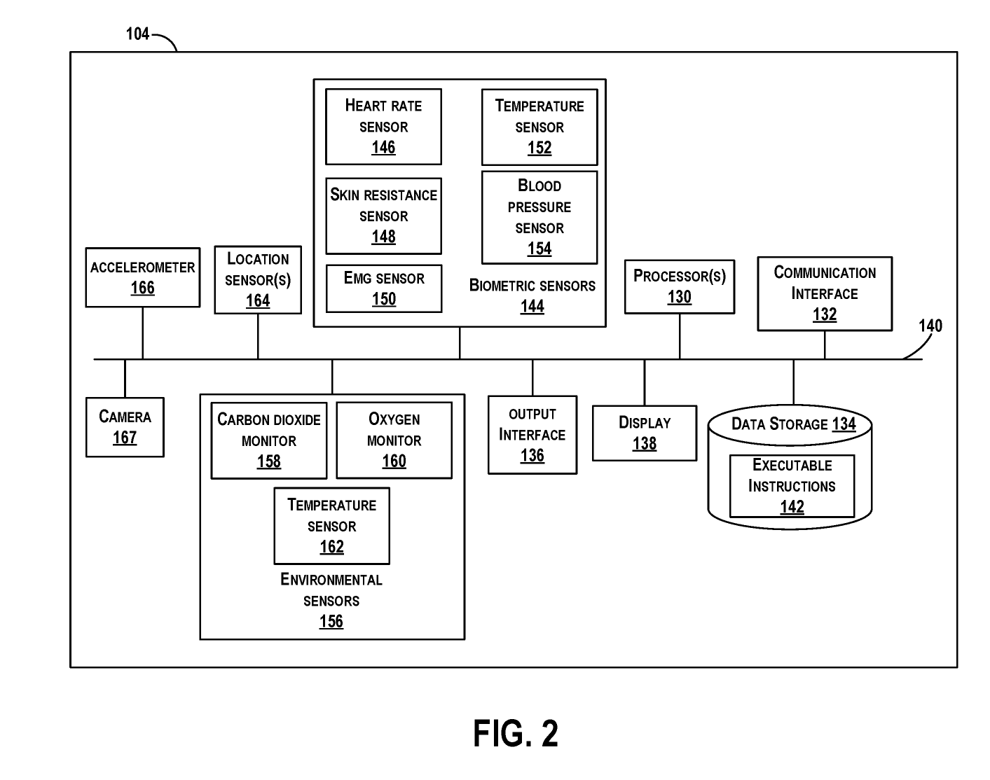

FIG. 2 is a block diagram of an example of the biometric interface 104, according to an example implementation. The biometric interfaces 104a-c may take the form of a wearable biometric interface such as a wristwatch, chest-band, necklace, etc. The biometric interface 104 includes a processor(s) 130, a communication interface 132, data storage 134, an output interface 136, and a display 138 each connected to a communication bus 140. The biometric interface 104 may also include hardware to enable communication within the biometric interface 104 and between the biometric interface 104 and other devices (not shown).

The hardware may include transmitters, receivers, and antennas, for example.

The communication interface 132 may be a wireless interface and/or one or more wireline interfaces that allow for both short-range communication and long-range communication to one or more networks or to one or more remote devices. Such wireless interfaces may provide for communication under one or more wireless communication protocols, such as Bluetooth, WiFi (e.g., an institute of electrical and electronic engineers (IEEE) 802.11 protocol), Long-Term Evolution (LTE), cellular communications, near-field communication (NFC), and/or other wireless communication protocols. Such wireline interfaces may include an Ethernet interface, a Universal Serial Bus (USB) interface, or similar interface to communicate via a wire, a twisted pair of wires, a coaxial cable, an optical link, a fiber-optic link, or other physical connection to a wireline network. Thus, the communication interface 132 may be configured to receive input data from one or more devices, and may also be configured to send output data to other devices. The communication interface 132 may also include a user-input device, such as a button or touchscreen, for example.

The data storage 134 may include or take the form of one or more computer-readable storage media that can be read or accessed by the processor(s) 130. The computer-readable storage media can include volatile and/or non-volatile storage components, such as optical, magnetic, organic or other memory or disc storage, which can be integrated in whole or in part with the processor(s) 130. The data storage 134 is considered non-transitory computer readable media. In some examples, the data storage 134 can be implemented using a single physical device (e.g., one optical, magnetic, organic or other memory or disc storage unit), while in other examples, the data storage 134 can be implemented using two or more physical devices.

The data storage 134 thus is a non-transitory computer readable storage medium, and executable instructions 142 are stored thereon. The instructions 142 include computer executable code. When the instructions 142 are executed by the processor(s) 130, the processor(s) 130 are caused to perform functions. Such functions include gathering or collecting data from sensors, and providing the data to the computing device 110, as shown in FIG. 1.

The processor(s) 130 may be a general-purpose processor or a special purpose processor (e.g., digital signal processors, application specific integrated circuits, etc.). The processor(s) 130 may receive inputs from the communication interface 132, and process the inputs to generate outputs that are stored in the data storage 134 and output to the display 138. The processor(s) 130 can be configured to execute the executable instructions 142 (e.g., computer-readable program instructions) that are stored in the data storage 134 and are executable to provide the functionality of the biometric interface 104 described herein.

The output interface 136 outputs information to the display 138 or to other components as well. Thus, the output interface 136 may be similar to the communication interface 132 and can be a wireless interface (e.g., transmitter) or a wired interface as well. The display 138 may be a touchscreen display to accept inputs as well.

The biometric interface 104 may further include a rechargeable lithium-polymer battery (not shown), and has a range of operating temperatures (4° to 113° F.) and non-operating temperatures (−22° to 140° F.). The biometric interface 104 further may have a maximum operating altitude (30,000 feet), and can be made to be water resistant and to have voice control, such as by including a microphone (not shown) to receive voice inputs that are processed by the processor(s) 130 into instructions.

The biometric interface 104 also includes biometric sensors 144 coupled to the communication bus 140. The biometric sensors 144 output data that may enable the computing device 110 to determine when a mechanic is experiencing fear, shock, or other trauma, for example. The biometric sensors 144 can output biometric data such as heart rate data, blood pressure data, galvanic skin resistance data, and skin temperature data. Thus, the biometric sensors 144 include a heart rate sensor 146, a skin resistance sensor 148, an electromyography (EMG) sensor 150, a temperature sensor 152, and a blood pressure sensor 154.

The heart rate sensor 146 detects a heart rate of the wearer. The heart rate sensor 146 may be a pulsometer or other electronic component that measures the heart rate both graphically and digitally (heartbeats per minute). The heart rate sensor 146 may alternatively be a heart rate monitor. A sudden increase in heart rate could indicate severe stress and consciousness. A sudden decrease in heart rate could indicate severe incapacitation or unconsciousness.

The skin resistance sensor 148 detects electrical conductance of skin of the wearer that varies according to moisture level. The skin resistance sensor 148 may detect a galvanic skin resistance (GSR) or skin conductance. Sweat glands are controlled by the sympathetic nervous system, and moments of strong emotion change an electrical resistance of the skin. Thus, skin conductance can be used as an indication of psychological or physiological excitation.

The EMG sensor 150 detects activity of muscles of the wearer. For example, EMG is a technique used to assess and record electrical activity produced by skeletal muscles, and EMG is recorded using an electromyograph, which detects action potential that activates muscle cells. An electromyogram measures the electrical activity of the muscles when the muscles are relaxed and being contracted.

The temperature sensor 152 detects a temperature of skin of the wearer. Skin temperature generally decreases when stressed or frightened, which can be a useful indication of fear of the wearer.

The blood pressure sensor 154 detects a blood pressure of the wearer.

The biometric interface 104 may transmit outputs of any and all of the biometric sensors 144 to the computing device 110, and the computing device 110 controls operation of the power unit 112 to pause movement of the one or more components 115 of the equipment 114 based on the received biometric data of the wearer being outside of a range of baseline data. An example range of baseline data may include normal heart rate, skin resistance, EMG, skin temperature, and blood pressure specific for the wearer, or as generally expected to be seen for a human of certain age, weight, and height that matches to the wearer.

The biometric interface 104 also includes environmental sensors 156 coupled to the communication bus 140. The environmental sensors 156 enable the computing device 110 to determine when a wearer is exposed to environmental dangers. The environmental sensors 156 include a carbon dioxide monitor 158, an oxygen monitor 160, and a temperature sensor 162.

The carbon dioxide monitor 158 detects amounts of carbon dioxide in an area of the wearer. Carbon dioxide is odorless and colorless, and thus, high levels of carbon dioxide in enclosed spaces can be dangerous.

The oxygen monitor 160 detects amounts of oxygen in an area of the wearer. Wherever hazardous gases or vapor-producing liquids are used, transported, or stored, there exists a possibility that such gases could accidentally leak into a surrounding area. A hazardous atmosphere is one that may expose mechanics to risks, and such hazardous atmospheres include flammable gas, vapor, or mist in excess of 10% of its lower flammability limit (LFL) or lower explosive limit (LEL) (e.g., the LFL/LEL is a lowest concentration of flammable gas or vapor that will ignite if an ignition source, such as spark, fire, static electricity discharge, etc. is present), a concentration of airborne combustible dust that meets or exceeds its LFL (approximated as the obscuring of vision at a distance of five feet or less), less than 19.5% or more than 23.5% percent oxygen by volume, a concentration of any toxic, corrosive, or asphyxiate substance above its “permissible exposure limit” (PEL), or any other atmospheric condition that is immediately dangerous to life or health. Based on levels of oxygen in the air, a hazardous atmosphere can be detected.

The temperature sensor 162 detects an ambient temperature of the wearable biometric interface 104. An ambient temperature sensor helps correct for mistakes, and outputs can be useful to determine, for example, when a mechanic enters a hot room rather than having a stressful reaction.

The biometric interface 104 may transmit outputs of any and all of the environmental sensors 156 to the computing device 110, and the computing device 110 controls operation of the power unit 112 to pause movement of the one or more components 115 of the equipment 114 based on the received data of the wearer being outside of a range of normal operating conditions. Normal operating conditions may vary based on a location of the work being performed, for example. In an example, data regarding normal operating conditions can be stored in the computing device 110 per location of an operating environment.

The biometric interface 104 also includes a location sensor(s) 164. The location sensor(s) 164 are used to determine position relative to dangerous zones. For example, location can be used to provide equivalent data of a pressure mat or could be used to determine when someone is physically located between a slat and a moving component of the equipment 114. Additional fixed position transmitters are used to provide increased accuracy. The location sensor(s) 164 can include a GPS with wide area augmentation system (WAAS) that is a system of satellites and ground stations that provide GPS signal corrections, giving better position accuracy than GPS by itself. WAAS corrects for GPS signal errors caused by ionospheric disturbances, timing, and satellite orbit errors, and it provides vital integrity information regarding the health of each GPS satellite. WAAS is generally five times more accurate than GPS by itself. A WAAS-capable receiver can give a position accuracy of better than 3 m at most times. When WAAS is augmented with local position transmitters, accuracy of 0.5 meters is possible. An optional network of local area position transceivers provides increased positional accuracy over WAAS enabled GPS, and a capability of providing positional data when GPS signals are weak or unavailable. Typical GPS position accuracy without WAAS is 15 m, and WAAS typical position accuracy is less than 3 m. By adding a network of local area position transceivers with known geographical locations relative to the work cell, accuracy can be increased to less than 0.5 m.

The biometric interface 104 also includes an accelerometer 166 for outputting data indicative of x,y,z-axis movement data of the wearable biometric interface 104. The accelerometer 166 thus provides 3-axis movement data and is used to determine if a wearer is or has fallen. Absolute stillness or sudden change in position or height could be an alert condition in a variety of scenarios. The computing device 110 can receive outputs of the accelerometer 166 and control operation of the power unit 112 to pause movement of the one or more components 115 of the equipment 114 based on the x,y,z-axis movement data of the wearable biometric interface being above a threshold.

The biometric interface 104 also includes a camera 167 for outputting images and video, which can include live-video, to the processor(s) 130.

Thus, the biometric interface 104 includes a plurality of biometric sensors 144 for sensing different biometric data of the wearer and environmental sensors 156 for sensing one or more characteristics of an environment of the wearable biometric interface 104. The computing device 110 can receive outputs from any and all of such sensors from the biometric interface 104 and control operation of the power unit 112 to pause movement of the one or more components 115 of the equipment 114 based on a combination of outputs of (i) the plurality of biometric sensors 144 being outside of the range of baseline data and (ii) the one or more environmental sensors 156 being outside of a range of normal operating conditions.

The biometric interface 104 can include further components as well, such as a battery monitor to provide cell balancing and accurate voltage and temperature monitoring to safeguard the batteries. A battery monitor calculates power consumed and a state of charge of the batteries, and transmits voltage, charge, discharge current, consumed power, and remaining battery capacity. The battery monitor is equipped with a programmable alarm to turn off devices when the battery voltage is below programmable boundaries. The battery monitor switches the physical primary battery and backup battery at each power up in order to balance battery use and maximize longevity. During normal operation, power is supplied only from the primary battery. The standby battery provides power whenever primary battery power is insufficient. This provides time for the biometric interface 104 to switch to a failsafe mode and log system parameters.

The biometric interface 104 includes an equipment ID that identifies the particular unit and is used to identify whether this unit has been registered as belonging to a local work cell. While the system 100 tracks and provides positional data on all transceivers detected in the area, the equipment ID allows the particular unit to utilize advanced features programmed for the local work cell.

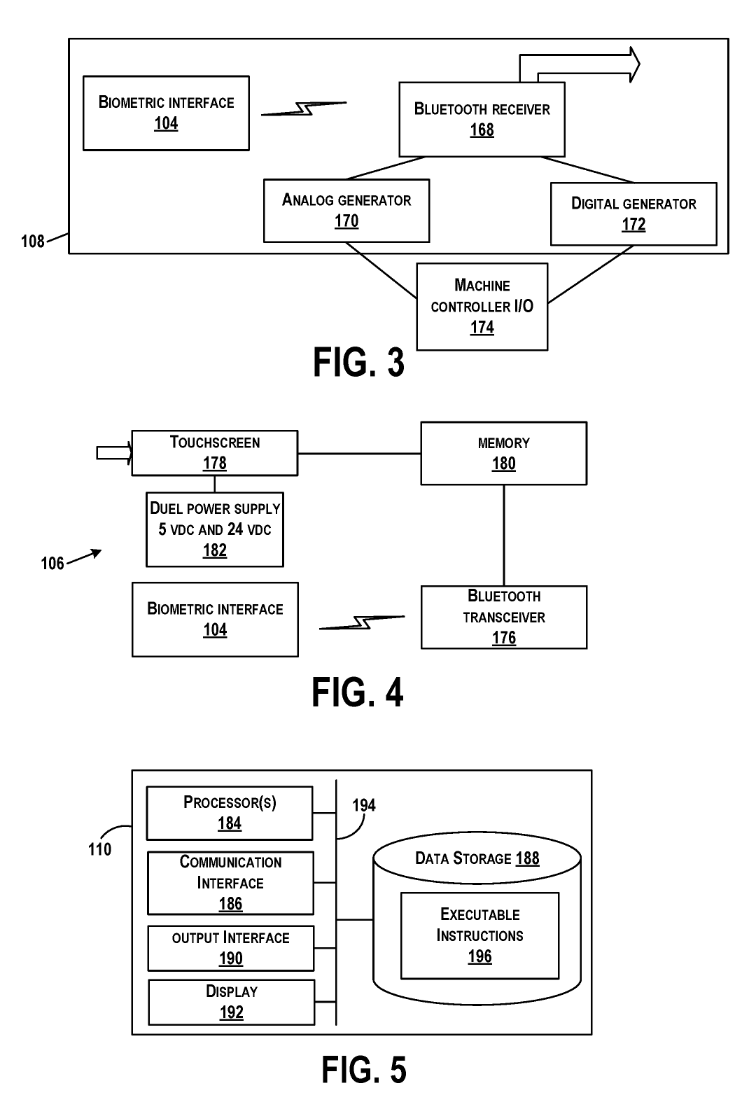

FIG. 3 is a block diagram of an example of the equipment interface 108, according to an example implementation. The equipment interface 108 is in communication with the biometric interface 104 through a wireless communication, and the equipment interface 108 receives interlock, stop/start status, and other data from the biometric interface 104. The equipment interface 108 then provides an interface between the biometric interface 108 and the existing equipment controllers, such as the computing device 110, for example, based on the received data.

The equipment interface 108 includes a Bluetooth receiver 168, an analog generator 170, a digital generator 172, and is in communication with a machine controller I/O 174. The equipment interface 108 converts Bluetooth data received at the Bluetooth receiver 168 (such as digital and analog biometric data signals) to digital/analog signals (via the analog generator 170 and digital generator 172) that can be read at the machine controller I/O 174.

The equipment interface 108 then transfers received biometric data to the computing device 110. Discrete wires can be used to wire the biometric signals from the equipment interface 108 to the computing device 110 where the biometric data is received and acted upon by application programs. For example, airplane test programmers can program biometric data functions such as stop, go, or back up into an airplane test program that is executed by the computing device 110.

FIG. 4 is a block diagram of an example of the central monitoring station 106, according to an example implementation. The central monitoring station 106 is also in communication with the biometric interface 104, such as through a Bluetooth transceiver 176, and the central monitoring station 106 receives data from the biometric interface 104, and can display and activate alarms in the system 100 based on the received data. The central monitoring station 106 is a computer station with a touchscreen 178, and receives data from each of the biometric interfaces 104a-c and displays a location of each wearer along with interlocks and start/stop status. Active alarms are displayed on the touchscreen 178 and announced via an audible alarm. The touchscreen 178 allows alarms to be silenced via a touch input. The central monitoring station 106 is also used for recording and transmitting status to the computing device 110. The central monitoring station 106 is also shown to include a memory 180 for storing status information, and a duel power supply (5 VDC and 24 VDC) 182 for supplying power.

FIG. 5 is a block diagram of an example of the computing device 110, according to an example implementation. The computing device 110 may be used to perform functions of methods described herein. The computing device 110 has a processor(s) 184, and also a communication interface 186, data storage 188, an output interface 190, and a display 192 each connected to a communication bus 194. The computing device 110 may also include hardware to enable communication within the computing device 110 and between the computing device 110 and other devices (not shown). The hardware may include transmitters, receivers, and antennas, for example.

The communication interface 186 may be a wireless interface and/or one or more wireline interfaces that allow for both short-range communication and long-range communication to one or more networks or to one or more remote devices. Such wireless interfaces may provide for communication under one or more wireless communication protocols, Bluetooth, WiFi (e.g., an institute of electrical and electronic engineers (IEEE) 802.11 protocol), Long-Term Evolution (LTE), cellular communications, near-field communication (NFC), and/or other wireless communication protocols. Such wireline interfaces may include an Ethernet interface, a Universal Serial Bus (USB) interface, or similar interface to communicate via a wire, a twisted pair of wires, a coaxial cable, an optical link, a fiber-optic link, or other physical connection to a wireline network. Thus, the communication interface 186 may be configured to receive input data from one or more devices, and may also be configured to send output data to other devices. The communication interface 186 may also include a user-input device, such as a keyboard or mouse, for example.

The data storage 188 may include or take the form of one or more computer-readable storage media that can be read or accessed by the processor(s) 184. The computer-readable storage media can include volatile and/or non-volatile storage components, such as optical, magnetic, organic or other memory or disc storage, which can be integrated in whole or in part with the processor(s) 184. The data storage 188 is considered non-transitory computer readable media. In some examples, the data storage 188 can be implemented using a single physical device (e.g., one optical, magnetic, organic or other memory or disc storage unit), while in other examples, the data storage 188 can be implemented using two or more physical devices.

The data storage 188 thus is a non-transitory computer readable storage medium, and executable instructions 196 are stored thereon. The instructions 196 include computer executable code. When the instructions 196 are executed by the processor(s) 184, the processor(s) 184 are caused to perform functions including controlling operation of the power unit 112 to pause movement of the one or more components 115 of the equipment 114.

The processor(s) 184 may be a general-purpose processor or a special purpose processor (e.g., digital signal processors, application specific integrated circuits, etc.). The processor(s) 184 may receive inputs from the communication interface 186, and process the inputs to generate outputs that are stored in the data storage 188 and output to the display 192. The processor(s) 184 can be configured to execute the executable instructions 196 (e.g., computer-readable program instructions) that are stored in the data storage 188 and are executable to provide the functionality of the computing device 110 described herein.

The data storage 188 may store baseline data, or ranges of baseline data for any outputs for any and all of the biometric sensors 144 and the environmental sensors 156 of the biometric interface 104. Such data may be predetermined or known, and pre-stored on the computing device 110, or can be determined per wearer based on certain age, weight, and height that matches to the wearer. Some examples of baseline data can include a blood pressure reading of a systolic pressure of less than 120 millimeters of mercury (mm Hg) and a diastolic pressure of less than 80 mm Hg, or 120/80 mm Hg, and a heart rate of between 60-80 beats per minute (bpm). Thus, a range of baseline data to be considered for blood pressure can include readings between about 80-120 mm Hg over 60-80 mm Hg for blood pressure, and between about 60-80 bpm for heart rate. Additional examples include a range of a temperature of skin of between about 33° C. and 37° C., for example. Other baseline data can be established and stored in the data storage 188 for use in comparison with received biometric data, for example.

The data storage 188 may also store data for normal operating conditions of an environment, such as a temperature, carbon dioxide level, and oxygen level as expected to be seen or experienced in the environment, for example. An example range of normal operating conditions can include temperatures in a range of between about 60° to 80° F., oxygen levels in a range of between about 19.5% and 22%, and carbon dioxide levels in a range of between about 250-1000 ppm.

The output interface 190 outputs information to the display 192 or to other components as well. Thus, the output interface 190 may be similar to the communication interface 186 and can be a wireless interface (e.g., transmitter) or a wired interface as well.

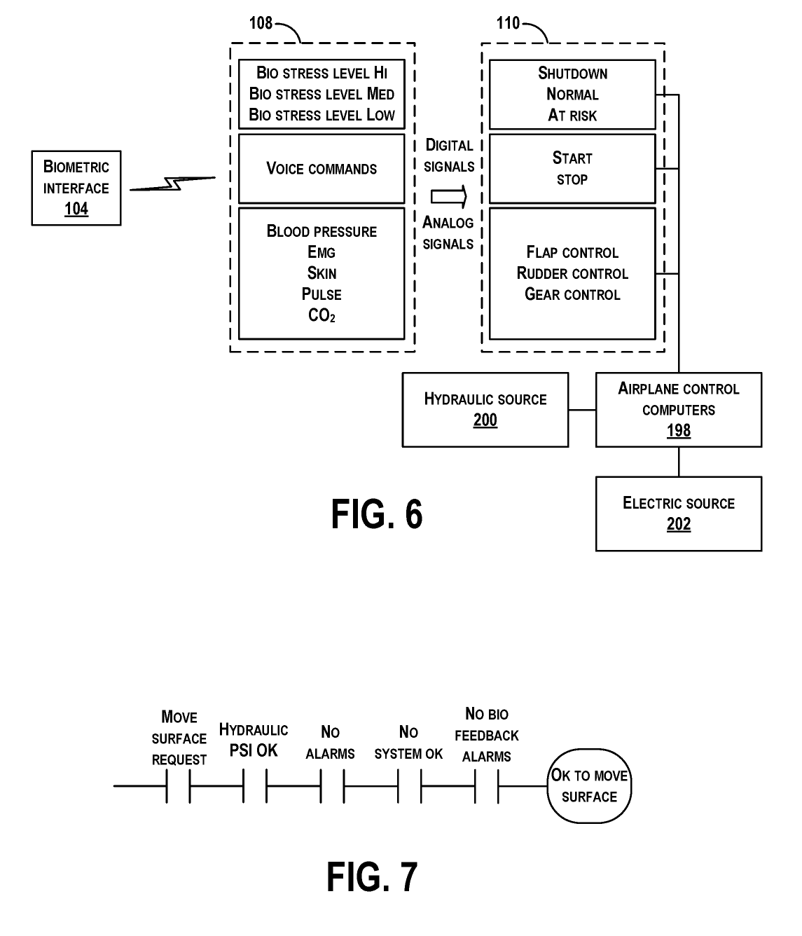

FIG. 6 is a block diagram illustrating an example operation and equipment interface 108 to computing device 110 connection, according to an example implementation. The equipment interface 108 receives interlock and/or start/stop status from each biometric interface 104a-c and provides the computing device 110 with program biometric data converted into a format for use in start/stop functions, such as for starting and stopping moving surfaces. As an example, the equipment interface 108 can receive wireless signals from the biometric interface 104, such as voice commands, blood pressure data, EMG data, skin resistance/temperature data, pulse data, carbon dioxide data, and can translate such received data into stress level readings of hi, med, and low, for example. Specific mappings of received biometric and environmental data to stress level readings can vary based on individual baseline numbers. In one example, for a blood pressure reading above normal, with high levels of CO₂ detected, a high stress level can be determined. In another example, for pulse readings above normal, and skin temperature above normal as well, a high stress level can be determined. In these examples, normal may be determined per individual based on age, height, weight, etc.

In further examples, when any one data output from any sensor is above normal, then a medium stress level can be determined, and when any two data points from two sensors are above normal, a high stress level can be determined.

The equipment interface 108 sends the data to the computing device 110, which is shown in FIG. 6 to operate airplane control computers 198 in turn to control a hydraulic source 200 and an electric source 202. The hydraulic source 200 and the electric source 202 control the hydraulic system 117 and the electrical system 116 in FIG. 1 for moving the components 115 of the equipment 114, for example. The computing device 110 can perform functions such as start/stop movement, or provide precise control for components such as flap control, rudder control, and gear control. In some examples, based on the stress level reading determined, the computing device 110 can move to shutdown the system (for a hi stress level), actuate an alarm (for a med stress level) or consider the system to be normal (for a low stress level).

FIG. 7 is a truth table illustrating an example of the computing device 110 processing the data for controlling operation of the equipment 114, according to an example implementation. In this example, a Move Surface request is issued and a check of the hydraulic source 200 is performed. When there are no alarms, and all system checks are OK, bio feedback alarms are determined based on the received biometric and environmental data from the biometric interface 104. In the example shown in FIG. 7, there are no bio feedback alarms, and thus, the surface movement can be initiated. In the example shown in FIG. 7, for the surface movement to occur, all items need to be determined to be “true”. In an alternative example, when a bio signal goes off, the Move Surface output goes off and stops the moving surface.

Within additional examples, as shown in FIG. 1, the position transceivers 118, 120, 122, and 124 provide positional data relative to work cell hazard zones. The transceiver network provides increased positional accuracy (e.g., position accuracy can be less than 0.5 m) over WAAS enabled GPS and a capability of providing positional data when GPS signals are weak or unavailable. The position transceivers 118, 120, 122, and 124 can also detect and locate other Bluetooth transmitters (e.g., cell phones) and two-way radios in the area. A multi-band radio scanner can further be included to monitor areas for active transmissions and when transmission is detected, it uses the local area position transceiver network to provide a position fix on the cell phone or radio.

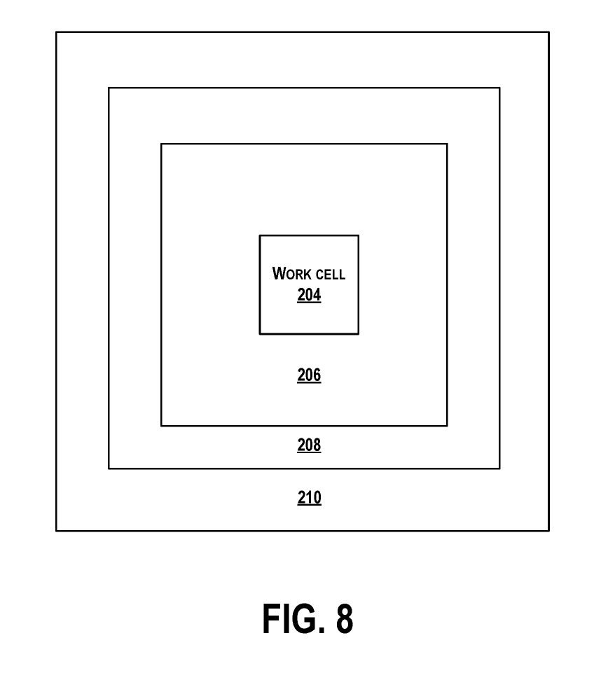

FIG. 8 is a block diagram illustrating an example work zone environment, according to an example implementation. In FIG. 8, a work cell 204 is an area in which equipment testing is being performed and moving components are present. Further physical areas surrounding the work cell 204 can be defined with geographic or location boundaries (e.g., x,y coordinates) as well, such as areas 206, 208, and 210. Each additional defined area may have an associated hazard warning, such as the area 206 defined as a hazard area, the area 208 defined as a warning area, and the area 210 defined as a caution area. Hazard area can be defined and programmed into the system 100 using the programming station 102, for example. The programming station 102 can also set up each of the biometric interfaces 104a-c to be restricted for use in certain areas or associated with specific personnel who work in certain areas, and then when or if the biometric interface 104 is determine to be outside of the preset area, an alarm can be triggered.

The work zone environment in FIG. 8 can then be used with the local position transceiver network (including the position transceivers 118, 120, 122, and 124) to determine position and transmit position and hazard warning data to the central monitoring station 106 and the equipment interface 108. In some examples, the computing device 110 can then receive location information of the wearable biometric interface 104a-c from the equipment interface 108, and based on the wearable biometric interface being within a predefined area, control operation of the power unit 112 to perform a safeguarding action relating to the movement of the one or more components 115 of the equipment 114. For example, when movement of a component in the work cell 204 prohibits personnel from being present in the work cell 204, based on detection of the biometric interface 104 in the work cell 204, the movement can be stopped for safety.

In another example, the computing device 110 can receive location information of one or more other wireless communication devices (e.g., device 126 or device 128), and based on the one or more other wireless communications devices being within a predefined area, control operation of the power unit 112 to perform a safeguarding action relating to the movement of the one or more components 115 of the equipment 114. In this example, when some unknown device is detected as being in the area 206 (e.g., hazard area) or the area 208 (e.g., warning area), the testing can be paused for safety of the personnel.

Thus, the computing device 110 can take into account location data of the biometric interface 104 and other devices, as well as biometric data output from the biometric interface 104 in order to determine whether to pause, start, or stop operation of the system test. The location information and biometric data can all be considered real-time data, or data currently collected and received for processing by the computing device 110 in real-time. Real-time processing can include performing functions in real-time during the system testing. These actions can then occur with no delay to process additional data received or through manual input. The real time processing means that the computing device 110 performs the actions of determining whether to pause operation of the equipment 114 during receipt of biometric data from one or more of the biometric interfaces 104a-c. The real time processing may continually process outputs of the biometric interfaces 104a-c to determine whether any output is indicative of a condition associated with one of the interruption scenarios.

The biometric interface 104a-c (and the biometric sensors 144 and the environmental sensors 156) can be considered to be always-on and always collecting data that is provided to the equipment interface 108 for processing.

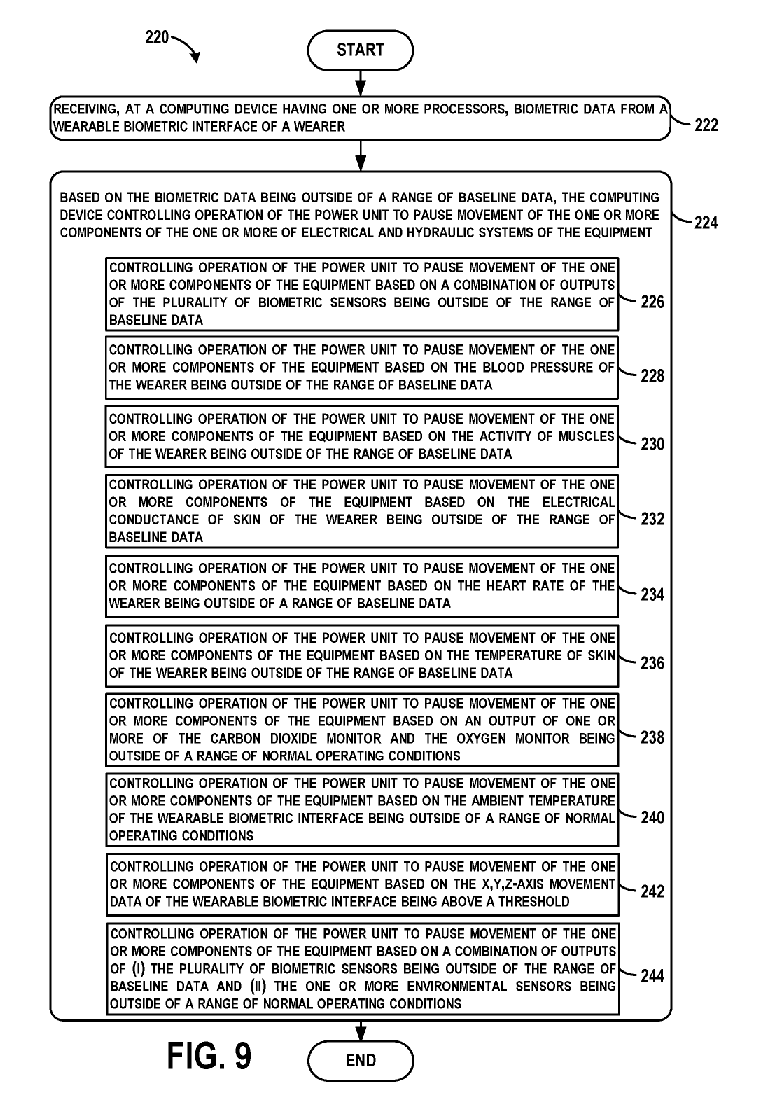

FIG. 9 shows a flowchart of an example method 220 for pausing operation of one or more of the electrical system 116 and the hydraulic system 117 of the equipment 114, according to an example implementation. Method 220 shown in FIG. 9 presents an example of a method that could be used with the system 100 or the computing device 110, shown in FIG. 1, for example. Further, devices or systems may be used or configured to perform logical functions presented in FIG. 9. In some instances, components of the devices and/or systems may be configured to perform the functions such that the components are actually configured and structured (with hardware and/or software) to enable such performance. In other examples, components of the devices and/or systems may be arranged to be adapted to, capable of, or suited for performing the functions, such as when operated in a specific manner. Method 220 may include one or more operations, functions, or actions as illustrated by one or more of blocks 222-224. Although the blocks are illustrated in a sequential order, these blocks may also be performed in parallel, and/or in a different order than those described herein. Also, the various blocks may be combined into fewer blocks, divided into additional blocks, and/or removed based upon the desired implementation.

It should be understood that for this and other processes and methods disclosed herein, flowcharts show functionality and operation of one possible implementation of present examples. In this regard, each block may represent a module, a segment, or a portion of program code, which includes one or more instructions executable by a processor for implementing specific logical functions or steps in the process. The program code may be stored on any type of computer readable medium or data storage, for example, such as a storage device including a disk or hard drive. Further, the program code can be encoded on a computer-readable storage media in a machine-readable format, or on other non-transitory media or articles of manufacture. The computer readable medium may include non-transitory computer readable medium or memory, for example, such as computer-readable media that stores data for short periods of time like register memory, processor cache and Random Access Memory (RAM). The computer readable medium may also include non-transitory media, such as secondary or persistent long term storage, like read only memory (ROM), optical or magnetic disks, compact-disc read only memory (CD-ROM), for example. The computer readable media may also be any other volatile or non-volatile storage systems. The computer readable medium may be considered a tangible computer readable storage medium, for example.

In addition, each block in FIG. 9, and within other processes and methods disclosed herein, may represent circuitry that is wired to perform the specific logical functions in the process. Alternative implementations are included within the scope of the examples of the present disclosure in which functions may be executed out of order from that shown or discussed, including substantially concurrent or in reverse order, depending on the functionality involved, as would be understood by those reasonably skilled in the art.

At block 222, the method 220 includes receiving, at the computing device 110 having one or more processors 184, biometric data from the wearable biometric interface 104 of a wearer. At block 222, the method 220 includes based on the biometric data being outside of a range of baseline data, the computing device 110 controlling operation of the power unit 112 to pause movement of the one or more components 115 of the one or more of electrical system 116 and the hydraulic system 117 of the equipment 114.

The biometric data can be useful to infer that a wearer may be in trouble or in danger, and further, with outputs from the environmental sensors 156, it can also be inferred that the wearer is in a dangerous location, and thus, the computing device 110 can prohibit system testing from taking certain actions. Anyone working on or near energized components or around airplane systems that have the possibility of movement, for example, can wear the biometric interface 104 to safeguard from being placed in danger, at which time, the computing device 110 can send instructions to de-energize and immediately stop movement of the components 115 from occurring.

The computing device 110 can control operation of the power unit 112 to enable or disable movement of the one or more components 115 of the equipment 114 based on the biometric data being outside of a range of baseline data.

The computing device 110 can control operations of the system testing based on many parameters or factors. Below are some examples.

Within one example, as shown at block 226, the wearable biometric interface 104 includes a plurality of the biometric sensors 144 for sensing different biometric data of the wearer, and the computing device 110 controls operation of the power unit 112 to pause movement of the one or more components 115 of the equipment 114 based on a combination of outputs of the plurality of the biometric sensors 144 being outside of the range of baseline data. As one example, based on outputs of the accelerometer 166 indicating someone is falling, in combination with outputs of the blood pressure sensor 154 indicating a high level outside of the baseline range, the computing device 110 can determine that the operation should be paused for safety of the wearer.

Within another example, as shown at block 228, the wearable biometric interface 104 includes the blood pressure sensor 154 for detecting a blood pressure of the wearer, and the computing device 110 controls operation of the power unit 112 to pause movement of the one or more components 115 of the equipment 114 based on the blood pressure of the wearer being outside of the range of baseline data.

Within another example, as shown at block 230, the wearable biometric interface 104 includes the EMG sensor 150 for detecting activity of muscles of the wearer, and the computing device 110 controlling operation of the power unit 112 to pause movement of the one or more components 115 of the equipment 114 based on the activity of muscles of the wearer being outside of the range of baseline data.

Within another one example, as shown at block 232, the wearable biometric interface 104 includes the skin resistance sensor 148 for detecting electrical conductance of skin of the wearer that varies according to moisture level, and the computing device 110 controls operation of the power unit 112 to pause movement of the one or more components 115 of the equipment 114 based on the electrical conductance of skin of the wearer being outside of the range of baseline data.

Within another example, as shown at block 234, the wearable biometric interface 104 includes the heart rate sensor 146 for detecting a heart rate of the wearer, and the computing device 110 controls operation of the power unit 112 to pause movement of the one or more components 115 of the equipment 114 based on the heart rate of the wearer being outside of a range of baseline data.

Within another example, as shown at block 236, the wearable biometric interface 104 includes the temperature sensor 152 for detecting a temperature of skin of the wearer, and the computing device 110 controls operation of the power unit 112 to pause movement of the one or more components 115 of the equipment 114 based on the temperature of skin of the wearer being outside of the range of baseline data.

Within another example, as shown at block 238, the wearable biometric interface 104 includes one or more environmental sensors 156 including one or more of the carbon dioxide monitor 158 and the oxygen monitor 160, and the computing device 110 controls operation of the power unit 112 to pause movement of the one or more components 115 of the equipment 114 based on an output of one or more of the carbon dioxide monitor 158 and the oxygen monitor 160 being outside of a range of normal operating conditions.

Within another example, as shown at block 240, the wearable biometric interface 104 includes the temperature sensor 162 for detecting an ambient temperature of the wearable biometric interface 104, and the computing device 110 controls operation of the power unit 112 to pause movement of the one or more components 115 of the equipment 114 based on the ambient temperature of the wearable biometric interface 104 being outside of a range of normal operating conditions.

Within another example, as shown at block 242, the wearable biometric interface 104 includes the accelerometer 166 for outputting data indicative of x,y,z-axis movement data of the wearable biometric interface 104, and the computing device 110 controls operation of the power unit 112 to pause movement of the one or more components 115 of the equipment 114 based on the x,y,z-axis movement data of the wearable biometric interface 104 being above a threshold.

Within another example, as shown at block 244, the wearable biometric interface 104 includes a plurality of the biometric sensors 144 for sensing different biometric data of the wearer and one or more environmental sensors 156 for sensing one or more characteristics of an environment of the wearable biometric interface 104, and the computing device 110 controls operation of the power unit 112 to pause movement of the one or more components 115 of the equipment 114 based on a combination of outputs of (i) the plurality of biometric sensors 144 being outside of the range of baseline data and (ii) the one or more environmental sensors 156 being outside of a range of normal operating conditions. Any combination may be used to trigger pausing the operations, for example.

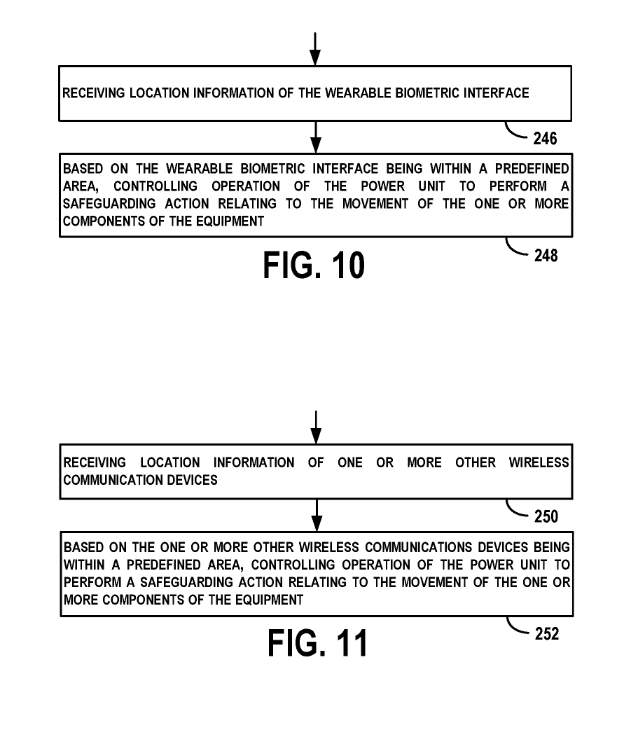

FIG. 10 shows a flowchart of an example method for use with the method 220, according to an example implementation. At block 246, functions include receiving location information of the wearable biometric interface 104, and at block 248, functions include based on the wearable biometric interface 104 being within a predefined area, controlling operation of the power unit 112 to perform a safeguarding action relating to the movement of the one or more components 115 of the equipment 114. The predefined area may be any of those as shown and described within FIG. 8, for example.

FIG. 11 shows a flowchart of an example method for use with the method 220, according to an example implementation. At block 250, functions include receiving location information of one or more other wireless communication devices (e.g., the device 126 and the device 128), and at block 252, functions include based on the one or more other wireless communications devices being within a predefined area, controlling operation of the power unit 112 to perform a safeguarding action relating to the movement of the one or more components 115 of the equipment 114.

By the term “substantially” and “about” used herein, it is meant that the recited characteristic, parameter, or value need not be achieved exactly, but that deviations or variations, including for example, tolerances, measurement error, measurement accuracy limitations and other factors known to skill in the art, may occur in amounts that do not preclude the effect the characteristic was intended to provide.

Different examples of the system(s), device(s), and method(s) disclosed herein include a variety of components, features, and functionalities. It should be understood that the various examples of the system(s), device(s), and method(s) disclosed herein may include any of the components, features, and functionalities of any of the other examples of the system(s), device(s), and method(s) disclosed herein in any combination, and all of such possibilities are intended to be within the scope of the disclosure.

The description of the different advantageous arrangements has been presented for purposes of illustration and description, and is not intended to be exhaustive or limited to the examples in the form disclosed. Many modifications and variations will be apparent to those of ordinary skill in the art. Further, different advantageous examples may describe different advantages as compared to other advantageous examples. The example or examples selected are chosen and described in order to best explain the principles of the examples, the practical application, and to enable others of ordinary skill in the art to understand the disclosure for various examples with various modifications as are suited to the particular use contemplated.

What is claimed is:

1. A method for pausing operation of one or more of electrical and hydraulic systems of an equipment, wherein a power unit is coupled to the equipment for enabling movement of one or more components of the one or more of electrical and hydraulic systems of the equipment, the method comprising:
receiving, at a computing device having one or more processors, biometric data from a wearable biometric interface of a wearer; and
based on the biometric data being outside of a range of baseline data, the computing device controlling operation of the power unit to pause movement of the one or more components of the one or more of electrical and hydraulic systems of the equipment.

2. The method of claim 1, wherein the computing device controlling operation of the power unit comprises controlling operation of the power unit to enable or disable movement of the one or more components of the equipment based on the biometric data being outside of a range of baseline data.

3. The method of claim 1, wherein receiving the biometric data from the wearable biometric interface comprises receiving the biometric data from the wearable biometric interface via a wireless communication.

4. The method of claim 1, wherein receiving the biometric data from the wearable biometric interface comprises receiving one or more of heart rate data, blood pressure data, galvanic skin resistance data, and skin temperature data.

5. The method of claim 1, wherein the wearable biometric interface comprises a plurality of biometric sensors for sensing different biometric data of the wearer, and
wherein controlling operation of the power unit to pause movement of the one or more components of the one or more of electrical and hydraulic systems of the equipment comprises controlling operation of the power unit to pause movement of the one or more components of the equipment based on a combination of outputs of the plurality of biometric sensors being outside of the range of baseline data.

6. The method of claim 1, wherein the wearable biometric interface comprises a blood pressure sensor for detecting a blood pressure of the wearer, and
wherein controlling operation of the power unit to pause movement of the one or more components of the one or more of electrical and hydraulic systems of the equipment comprises controlling operation of the power unit to pause movement of the one or more components of the equipment based on the blood pressure of the wearer being outside of the range of baseline data.

7. The method of claim 1, wherein the wearable biometric interface comprises an electromyography (EMG) sensor for detecting activity of muscles of the wearer, and
wherein controlling operation of the power unit to pause movement of the one or more components of the one or more of electrical and hydraulic systems of the equipment comprises controlling operation of the power unit to pause movement of the one or more components of the equipment based on the activity of muscles of the wearer being outside of the range of baseline data.

8. The method of claim 1, wherein the wearable biometric interface comprises a skin resistance sensor for detecting electrical conductance of skin of the wearer that varies according to moisture level, and
wherein controlling operation of the power unit to pause movement of the one or more components of the one or more of electrical and hydraulic systems of the equipment comprises controlling operation of the power unit to pause movement of the one or more components of the equipment based on the electrical conductance of skin of the wearer being outside of the range of baseline data.

9. The method of claim 1, wherein the wearable biometric interface comprises a heart rate sensor for detecting a heart rate of the wearer, and
wherein controlling operation of the power unit to pause movement of the one or more components of the one or more of electrical and hydraulic systems of the equipment comprises controlling operation of the power unit to pause movement of the one or more components of the equipment based on the heart rate of the wearer being outside of a range of baseline data.

10. The method of claim 1, wherein the wearable biometric interface comprises a temperature sensor for detecting a temperature of skin of the wearer, and
wherein controlling operation of the power unit to pause movement of the one or more components of the one or more of electrical and hydraulic systems of the equipment comprises controlling operation of the power unit to pause movement of the one or more components of the equipment based on the temperature of skin of the wearer being outside of the range of baseline data.

11. The method of claim 1, wherein the wearable biometric interface comprises one or more environmental sensors including one or more of a carbon dioxide monitor and an oxygen monitor, and
wherein controlling operation of the power unit to pause movement of the one or more components of the one or more of electrical and hydraulic systems of the equipment comprises controlling operation of the power unit to pause movement of the one or more components of the equipment based on an output of one or more of the carbon dioxide monitor and the oxygen monitor being outside of a range of normal operating conditions.

12. The method of claim 1, wherein the wearable biometric interface comprises a temperature sensor for detecting an ambient temperature of the wearable biometric interface, and
wherein controlling operation of the power unit to pause movement of the one or more components of the one or more of electrical and hydraulic systems of the equipment comprises controlling operation of the power unit to pause movement of the one or more components of the equipment based on the ambient temperature of the wearable biometric interface being outside of a range of normal operating conditions.

13. The method of claim 1, wherein the wearable biometric interface comprises an accelerometer for outputting data indicative of x,y,z-axis movement data of the wearable biometric interface, and
wherein controlling operation of the power unit to pause movement of the one or more components of the one or more of electrical and hydraulic systems of the equipment comprises controlling operation of the power unit to pause movement of the one or more components of the equipment based on the x,y,z-axis movement data of the wearable biometric interface being above a threshold.

14. The method of claim 1, wherein the wearable biometric interface comprises a plurality of biometric sensors for sensing different biometric data of the wearer and one or more environmental sensors for sensing one or more characteristics of an environment of the wearable biometric interface, and
wherein controlling operation of the power unit to pause movement of the one or more components of the one or more of electrical and hydraulic systems of the equipment comprises controlling operation of the power unit to pause movement of the one or more components of the equipment based on a combination of outputs of (i) the plurality of biometric sensors being outside of the range of baseline data and (ii) the one or more environmental sensors being outside of a range of normal operating conditions.

15. The method of claim 1, further comprising:
receiving location information of the wearable biometric interface; and
based on the wearable biometric interface being within a predefined area, controlling operation of the power unit to perform a safeguarding action relating to the movement of the one or more components of the equipment.

16. The method of claim 15, further comprising:
receiving location information of one or more other wireless communication devices; and
based on the one or more other wireless communications devices being within a predefined area, controlling operation of the power unit to perform a safeguarding action relating to the movement of the one or more components of the equipment.

17. A system comprising:
a wearable biometric interface for sensing biometric data of a wearer;
a power unit coupled to equipment for enabling movement of one or more components of the equipment; and
a computing device having one or more processors, the computing device in communication with the power unit, wherein the computing device receives the biometric data from the wearable biometric interface and controls operation of the power unit, based on the biometric data, for operating one or more of electrical and hydraulic systems of the equipment, wherein the computing device controls operation of the power unit to pause movement of the one or more components of the equipment based on the biometric data being outside of a range of baseline data.

18. The system of claim 17, wherein the computing device further includes data storage storing instructions executable by the one or more processors for controlling operation of the power unit for operating of the one or more electrical and hydraulic systems of the equipment based on the biometric data.

19. The system of claim 17, wherein the equipment includes an airplane, and wherein the one or more components include a surface of the airplane and landing gear of the airplane.

20. A system comprising:
a wearable biometric interface having a plurality of biometric sensors for sensing different biometric data of a wearer and one or more environmental sensors for sensing one or more characteristics of an environment of the wearable biometric interface; and
a computing device, having one or more processors, being in communication with a power unit that is coupled to an equipment for enabling movement of one or more components of the equipment, wherein the computing device receives the biometric data and the one or more characteristics of the environment from the wearable biometric interface and controls operation of the power unit to pause movement of the one or more components of the equipment based on a combination of outputs of (i) the plurality of biometric sensors being outside of a range of baseline data and (ii) the one or more environmental sensors being outside of a range of normal operating conditions.
